(19) United States
(12) Patent Application Publication
(10) Pub. No.: US 2011/0129932 A1
(43) Pub. Date: Jun. 2, 2011

(54) Lipoprotein Assay

(75) Inventors: Gareth Royston Jones, Altrincham (GB); David Thomas Clarke, Warrington (GB)

(73) Assignee: L3 TECHNOLOGY LIMITED, Herefordshire (GB)

(21) Appl. No.: 12/308,549

(22) PCT Filed: Jun. 12, 2007
(86) PCT No.: PCT/GB2007/002177

§ 371 Date: Dec. 15, 2008

(30) Foreign Application Priority Data
Jun. 15, 2006 (GB) 0611857.4

(51) Int. Cl. G01N 33/92

(52) U.S. Cl. 436/71

(57) ABSTRACT

The present invention concerns a method of determining the concentration of total lipoprotein in a sample. The method involves the steps of: (i) adding to an aliquot of the sample a lipophilic dye that binds to lipoproteins in the sample and which when so bound fluoresces under appropriate excitation; and (ii) determining the total lipoprotein concentration in the sample using fluorescence analysis. A method of analysing the lipoprotein content of a sample solution using a dye that discriminates between different types of lipoprotein is also disclosed.

The present invention relates to an assay system for determining the concentration of lipoproteins in samples such as blood plasma or serum. In one aspect of the invention the assay system may involve additional steps that may be used to discriminate between different classes of lipid molecules in a sample mixture.

Lipids are a diverse group of organic compounds occurring in living organisms. They are insoluble in water, but soluble in organic solvents. Lipids are broadly classified in to two categories: (i) complex lipids; and (ii) simple lipids. Complex lipids are esters of long-chain fatty acids and include glycerides, glycolipids, phospholipids, cholesterol esters and waxes. Simple lipids, which do not contain fatty acids, include steroids (for example, cholesterol) and terpenes.

Lipids can combine with proteins to form lipoproteins, which is the form in which lipids, such as cholesterol and triglycerides, are transported in blood and lymph. The lipoproteins found in blood plasma fall into three main classifications: (i) high density lipoproteins (HDL); (ii) low density lipoproteins (LDL); and (iii) very low density lipoproteins (VLDL), together with intermediate density lipoproteins (IDL).

It is well documented that there is a strong relationship between the concentration of the various lipoproteins in blood plasma and the risk of atherosclerosis, i.e. the development of harmful plaques on blood vessel walls, which can lead to a heart attack. It is also known that the different classes of lipoproteins (HDL, LDL and VLDL) each play a different role in atherosclerosis. For instance, HDL is regarded as being anti-atherogenic whereas LDL is known to be highly atherogenic (the cholesterol it carries correlating closely with atheroscleroses development).

Therefore, knowledge of the total lipoprotein content and also relative concentrations of each of the various lipid components in the blood (i.e. the lipoproteins) would be advantageous, as this would assist a clinician in treating patients having blood concentrations of these lipids, which are inappropriate. It will be appreciated that having a knowledge of the patient's lipoprotein profile would be most advantageous to the clinician.

Assays have been developed for determining the concentrations of some of the lipid components in blood. Such assays normally involve initially taking a blood sample from a patient, which is then sent to a clinical laboratory for analysis. Such assays are carried out using expensive equipment and for logistical reasons a considerable length of time is taken to generate results. This delays treatment. Furthermore, the tests are involved and are therefore expensive. In addition, the equipment used in the lab is not readily portable and so cannot be used by General Practitioners (GPs), or nurses, carrying out house calls, or even as test kits for home use. Devices have recently been developed that attempt to reproduce lab assays at “point of care” but these have proved to be expensive and require an expert user to operate. Accordingly, there is a requirement to provide improved methods for analysing the lipoprotein profile in blood serum.

Blood serum is a complex mixture of a variety of proteins, and although methods for separating and directly measuring the concentration of the different classes of lipoproteins are known, such methods are complex and expensive. An example of an assay for determining the lipoprotein concentration of blood serum is disclosed in WO 01/53829A1. This document relates to the use of a particular organic luminophore, 4-dimethylamino-4′-difluoromethyl-sulphonyl-benzylidene-acetophenone (DMSBA), as a fluorescent probe. The formula of the probe, identified as K-37, is given below:—

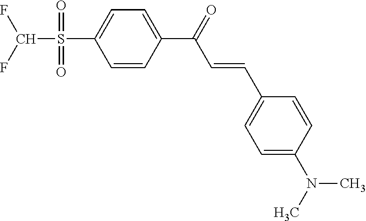

The probe K-37 is not luminous in water, but is highly luminous in aqueous lipoprotein solutions, such as blood serum. In particular, the intensity of the fluorescence is highly dependent upon the lipoprotein content of the blood serum and thus K-37 can be used as a fluorescent probe to measure the concentration of lipoproteins that may be present, i.e. K-37 fluoresces when bound to the lipids of lipoproteins and is excited at appropriate radiation wavelengths. Accordingly, measurement of the time-resolved fluorescence decay of a lipoprotein mixture can be used to give direct information as to the relative concentrations of the different lipoproteins (LDL, and VLDL) present in that mixture.

However, problems with using K-37 time-resolved fluorescence decay is that its measurement is complex and requires expensive equipment. Furthermore, it involves highly technical computer analysis of the data produced, which can be time-consuming to interpret correctly. Accordingly, use of K-37 time-resolved fluorescence decay to determine the concentrations of lipid components in blood has serious limitations for a clinician when wishing to quickly decide a course of treatment rather than taking the time to use time-resolved fluorescence decay to provide a lipoprotein analysis.

Therefore, even though there are methods available for determining the concentration of specific lipoproteins in a sample, by using time-resolved fluorescence analysis with the probe K-37, it will be appreciated that this method has a number of limitations.

It is therefore an aim of embodiments of the present invention to obviate or mitigate the problems with the prior art, and to provide an improved method for determining the concentration of lipoproteins in a sample.

The inventors decided to investigate whether or not they could develop a simplified assay based on the use of fluorescent dyes for measuring lipoproteins in a biological sample. This decision was made in view of a technical prejudice against investigating this sort of assay. Biological samples, and particularly blood samples, contain molecules that autofluoresce across a relatively broad wavelength and it was therefore expected that it would not be possible to develop a simple fluorescence based assay.

For determining the concentration of total lipoprotein (i.e. HDL, LDL, and VLDL) in a blood sample, the inventors realised that it would be preferred that the fluorescence response from a dye bound to the various lipoprotein classes must be substantially the same for a given total lipoprotein concentration, i.e. total lipoprotein concentration, irrespective of its composition (i.e. the ratio of HDL:LDL:IDL:VLDL in the sample). Accordingly, the inventors believed that it would be preferred that the response of fluorescence intensity from the dye substance should also be substantially linear across the range of concentrations of lipoprotein molecules that would be expected from samples that would be encountered in clinical tests.

While the inventors do not wish to be bound by any hypothesis, they believe that the intensity of fluorescence from the dye substance will depend on its affinity for a particular lipoprotein molecule (HDL, LDL, IDL or VLDL) in the sample, the quantum yield of fluorescence depending on the environment within that lipoprotein molecular complex, and also the degree of fluorescence quenching caused by energy transfer between dye molecules packed closely together. Hence, the inventors reasoned that it may be possible to select a suitable dye substances that may be used to make an accurate measurement of total lipoprotein by simple fluorescent measurement.

The inventors therefore conducted a series of experiments (discussed in Examples 1-3) to investigate whether it was possible to obtain a linear and equal relationship between the fluorescence of a number of dyes, and the lipoprotein concentration for each lipoprotein particle type (HDL, LDL, and VLDL), across a range of lipoprotein concentrations that would be encountered in real serum samples. To their surprise, they found that, for a particular class of dyes there was a linear relationship between fluorescence and lipoprotein concentration.

Hence, according to a first aspect of the present invention, there is provided a method of determining the concentration of total lipoprotein in a sample, the method comprising the steps of:—
(i) adding to an aliquot of the sample a lipophilic dye that binds to lipoproteins in the sample and which when so bound fluoresces under appropriate excitation; and
(ii) determining the total lipoprotein concentration in the sample using fluorescence analysis.

By the term “total lipoprotein”, we mean the collective concentration of VLDL, HDL, LDL, IDL and chylomicrons in the sample.

The inventors have established that fluorescent, lipophilic dyes may be advantageously used to determine total lipoprotein content of a sample. They have surprisingly found that such dyes overcome the shortcomings of prior art techniques and may be used in an accurate, quick and simple fluorescence based assay that may be conducted on simple fluorimeters that may be used in the field (e.g in a shop, GP's surgery or on a home visit) and do not require expert knowledge to operate.

A wide variety of lipophilic dyes may be used. However the inventors have established that biphenolic dyes (i.e. dyes comprising two phenol groups) are particularly useful according to the invention. Biphenolic dyes according to the first aspect of the invention may comprise two phenol groups separated by a carbon chain comprising at least three carbon atoms. The carbon chain also preferably comprises at least one unsaturated bond. It will be appreciated that such dyes may have substitutions on the phenol groups and the carbon chain.

In a preferred embodiment of the invention it is preferred that the dye substance comprises a fluorescent unit (i.e. a fluorescent chemical moiety) known as a chalcone or benzalideneacetophenone. This unit has the general formula:

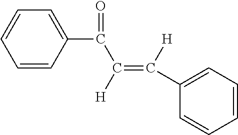

Chalcone or benzalideneacetophenone dyes have functional groups substituted on to the phenolic rings of the chalcone group. The nature of these functional groups can have a number of effects on the properties of the dye which include:
(a) shifting the excitation and emission wavelengths to longer wavelength;
(b) impart the advantage that the wavelength used to excite the dye will produce little background fluorescence from the many ultraviolet
(wavelengths <400 nm) excitable components of blood plasma;
(c) sensitivity to polarity of solvent (environment).
(d) charge transfer effects resulting in spectral changes; and
(e) quenching in non-polar solvents by intersystem crossing to the triplet state.

It will be appreciated that K-37 is an example of a substituted chalcone dye. Our co-pending, unpublished application PCT/GB2005/004794 concerns an improved assay based on K-37. Accordingly, in some embodiments of the first aspect of the invention, reference to chalcone based dyes, with regards the first aspect of the invention, is intended to preclude K-37. However when K-37 is employed according to the first aspect of the invention it is preferred that it is used as discussed below and as discussed in connection with the second aspect of the invention.

4-Dimethylamino methylchalcone may also be used according to this embodiment of the first aspect of the invention. This dye has the following formula:

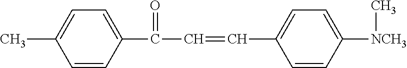

4-Dimethylamino methylchalcone comprises a chalcone unit with the para addition of a dimethylamino group and a methyl group.

The excitation maximum for 4-Dimethylamino methylchalcone is 420 nm and the emission maximum is 490 nm. It will therefore be appreciated that these wavelengths make the dye particularly suitable for assaying serum or plasma samples.

In another preferred embodiment of the first aspect of the invention the lipophilic dye is a dye substance comprising a fluorescent unit (i.e. a fluorescent chemical moiety) with the chemical structure: Ph-[C—C═C]ₙ—C-Ph. n may preferably from 2-6.

Preferred dyes comprising such a fluorescent unit are Diphenylhexatriene (DPH) and Diphenyloctatetrene (DPO).

DPH has the general formula:

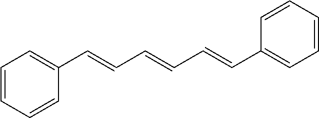

DPO has the general formula:

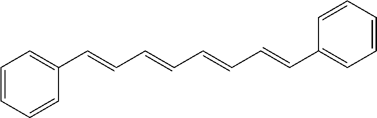

Ph-[C—C═C]ₙ—C-Ph dyes may have substitutions on the phenolic rings. As was the case for chalcone based dyes, these substitutions may regulate the fluorescent properties of the dye substance.

Ph-[C—C═C]ₙ—C-Ph dyes according to the invention may also be substituted on the carbon chain.

Other preferred Ph-[C—C═C]ₙ—C-Ph dyes include derivatives, that are known to the art, and are available with membrane components covalently bound to them (e.g. cholesterol, phospholipids, triglycerides, sphingomyelin etc.)

DPH has an excitation maximum at about 380 nm and emission maximum at 440 nm although it is excitable to about 400 mm. It will therefore be appreciated that DPH based dyes are suitable for use according to the invention because it is possible to avoid much of the contaminating fluorescence background associated with blood based samples (serum or plasma) by exciting at about 400 nm and reading at 440 nm.

DPO has similar properties to DPH but is even more preferred because it has excitation maximum at about 430 nm.

In a further preferred embodiment of the first aspect of the invention the lipophilic dye is a Coumarin dye or a derivative thereof. Such dyes are well known to the art.

A preferred Coumarin dye is Coumarin 30 which has the following structure:

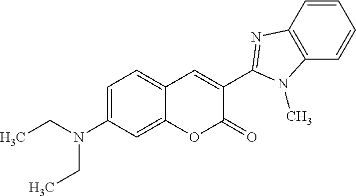

Coumarin 30 advantageously has the following characteristics:
1. a low fluorescence in PBS (phosphate buffered saline)
2. a low fluorescence in delipified plasma and therefore protein
3. a very high fluorescence in lipids

Suitably, the concentration of the lipophilic dye added to the sample may be between approximately 0.01-20.0 mM, more suitably, between approximately 0.05-10 mM, and even more suitably, between approximately 0.1-1.6 mM. It will be appreciated that the most preferred dye concentration will be unique to the dye used.

By way of example when the dye is a chalcone dye, the concentration of K-37 dye added to the sample may be between approximately 0.2-1.0 mM, more suitably, between approximately 0.3-0.9 mM, and even more suitably, between approximately 0.5-0.8 mM. Preferably, the concentration of K-37 added to the sample is between approximately 0.65-0.75 mM. 0.65 mM K-37 is an especially preferred concentrantion.

By way of further example when the dye is a Ph-[C—C═C]ₙ—C-Ph based dye, the concentration of dye added to the sample may be between approximately 0.01-20 mM (depending on the specific dye used). For instance when the dye is unsubstituted DPH, 0.05-5 mM is a preferred concentration and an especially preferred concentration is about 0.4 mM DPH. Alternatively when the dye is a DPO, the concentration of dye added to the sample may be between approximately 0.1-5.0 mM, more suitably, between approximately 0.2-1.0 mM, and even more suitably, between approximately 0.3-0.7 mM. Preferably, the concentration of DPO added to the sample is approximately 0.4-0.5 mM.

It will be appreciated that the method according to the first aspect of the invention comprises exciting the sample at an excitation wavelength and then observing the fluorescence at another emission wavelength. The choice of excitation and emission wavelength will depend on the properties of the chosen dyes.

A number of conventional fluorimetric devices may be used for the purposes of the present invention. A skilled person will appreciate that apparatus for determining the concentration of total lipoprotein in a sample may comprise a reaction reservoir for conducting a lipoprotein assay; containment means adapted to contain reagents required for the method according to the first aspect of the invention; excitation means operable to excite the sample so that it fluoresces (e.g. a light source such as diodes emitting light at desired wavelengths in conjunction with any required filters), and detection means (e.g. a photodiode or photomultiplier which is preferably yellow-red sensitive) operable to detect the fluorescence emitted by the sample.

The excitation and emission wavelengths of preferred dyes avoid the autofluorescence caused by components of blood samples (which can be interfering below about 300 nm.

Generally the excitation wavelengths for the dyes should be between about 350 nm-500 nm, and more preferably between about 400 nm-470 nm.

The method of the first aspect of the invention may comprise observing the fluorescence at an emission wavelength of above about 400 nm, and more preferably, at or above about 440 nm (e.g. 440 nm, 490 nm or 550 nm).

In a preferred embodiment, utilising K-37 the method comprises exciting the sample at an excitation wavelength of between about 400 nm-500 nm, and more preferably, between about 420 nm-480 nm, and even more preferably, between about 440 nm-470 nm. An especially preferred excitation wavelength of about 450 nm may be used although excitation at any wavelength between about 450-470 nm is also particularly preferred. Preferably, the method comprises observing the fluorescence at an emission wavelength of between about 500-650 nm, and more preferably, between about 520 nm-600 nm. An especially preferred emission wavelength of about 540 nm (or higher) may be used, at which the most accurate readings for determining the total lipoprotein concentration (i.e. the concentration of HDL, IDL, LDL and VLDL, but also chylomicrons if present) may be observed.

In another preferred embodiment, utilising 4-Dimethylamino methylchalcone, the excitation wavelength may be about 420 nm and the emission wavelength about 490 nm.

In another preferred embodiment, utilising DPH, the excitation wavelength may be 350-400 nm (and preferably about 400 nm) and the emission wavelength about 440 nm.

By the term “fluorescence analysis”, we mean the measurement of fluorescence of the products of the lipoprotein assay, by first exciting the sample so that it fluoresces, and then observing the fluorescence.

The sample may be a foodstuff, for which knowledge of the total lipoprotein concentration therein is required. Preferably, the sample is a biological sample, which may be obtained from a subject to be tested. The sample may comprise any biological fluid, for example, blood plasma or serum, or lymph. It is especially preferred that the sample comprises blood serum or plasma.

The sample may be diluted such that an expected concentration of total lipoprotein in the sample will be in the region of between approximately 0.1-50.0 mM, more suitably, between approximately 0.5-20 mM, and even more suitably, between approximately 1-10 mM. The skilled person will appreciate that the purpose of the assay is to measure the lipoprotein content although experience will also dictate that such a skilled person will be able to predict the range of concentrations they would expect to be found in a chosen sample. Accordingly, depending on the origin of the sample being tested, a person conducting the assay may chose to dilute the sample (e.g. with Phosphate Buffered Saline or a similar buffer) before conducting the assay. However, in preferred embodiments of the invention, it is possible to directly introduce a sample (e.g. serum) into the assay without needing to make any dilution. This has the advantage that the assay procedure may be kept simple and may be easily used in the field.

The inventors realised that the lipoprotein profile that may be generated using the method according to the first aspect of the invention may be further improved and more detailed, if they could distinguish between the various lipoproteins in the sample being tested. Therefore, the inventors investigated the use of probe substances other than the lipophilic dyes discussed above to see if it was possible to distinguish between the various lipoprotein molecules. They were surprised to find that a number of dyes, defined herein as discriminating dyes, are available that will bind to lipoproteins and will exhibit different fluorescent responses that are dependant on the particular lipoprotein bound. Fluorescent measurements with these dyes makes it possible to distinguish between the types of lipoprotein present in a sample. This is done by comparing the enhanced or reduced fluorescence caused by one specific type of lipoprotein in a lipoprotein mixture with the fluorescence expected from the other lipoproteins (in the absence of the one specific type of lipoprotein) as determined from a calibration curve and a known value of the total lipoprotein content given by the assay according to the first aspect of the invention. For example the inventors describe below how they found that the fluorescent dye, Nile Red, exhibited a significantly higher fluorescence in HDL than in the other lipoproteins, such as LDL and VLDL. Therefore, the inventors realised that a discriminating dye may be used to discriminate between classes or subclasses of lipoproteins in the sample. This is possible after the total lipid concentration has been determined according to the first aspect of the invention.

Hence, according to a second aspect of the present invention, there is provided a method of analysing the lipoprotein content of a sample solution, the method comprising the steps of:—
(a) adding to a first aliquot of the sample a lipophilic dye that binds to lipoproteins in the sample and which when so bound fluoresces under appropriate excitation;
(b) determining the total lipoprotein concentration in the first aliquot using fluorescence analysis;
(c) adding to a second aliquot of the sample a discriminating dye that binds to a specific lipoprotein or lipoproteins in the sample and which when so bound fluoresces under appropriate excitation;
(d) determining the concentration of the lipoproteins in the second aliquot using fluorescence analysis; and
(e) calculating the lipoprotein content by comparing the concentrations determined in steps (b) and (d).

Steps (c) and (d) of the method of the second aspect of the invention may be used to determine the concentration of a particular class, or sub-class of lipoprotein by the shift in fluorescence response of a dye specific to a particular lipoprotein.

Steps (a) and (b) of the method according to the second aspect of the invention may correspond to steps (i) and (ii) of the method according to the first aspect of the invention. Accordingly any lipophilic dye according to the first aspect of the invention may be employed according to the second aspect of the invention.

In one embodiment of the invention it is preferred that the discriminating dye is a dye other that Nile Red when step (a) utilises the lipophilic dye K-37.

In a preferred embodiment of the second aspect of the invention the lypophilic dye is K-37. K-37 may be used at a number of concentrations in step (a). However the inventors have found it is advantageous to use the dye at the concentration of 0.1-1.0 mM K-37. These concentrations are optimal for a more accurate determination of the concentration of the total lipoprotein. There is surprisingly considerably less signal distortion obtained from analysis of the fluorescence measurement of step (b) at these concentrations. It is preferred that the concentration of K-37 added to the sample may be between approximately 0.2-1.0 mM, more suitably, between approximately 0.3-0.9 mM, and even more suitably, between approximately 0.5-0.8 mM. Preferably, the concentration of K-37 added to the sample is between approximately 0.65-0.75 mM. 0.65 mM K-37 is an especially preferred concentration.

Hence, in a preferred embodiment, approximately 0.65 mM or 0.7 mM of the probe substance, K-37, is added to the sample in step (a) of the method in order to carry out step (b) of the method according to the second aspect of the invention.

When K-37 is used, step (a) of the method according to the second aspect comprises exciting the sample at an excitation wavelength of between about 400 nm-500 nm, and more preferably, between about 420 nm-480 nm, and even more preferably, between about 440 nm-470 nm. An especially preferred excitation wavelength of about 450 nm may be used although excitation at any wavelength between about 450-470 nm is also particularly preferred. Preferably, the method comprises observing the fluorescence at an emission wavelength of between about 500-650 nm, and more preferably, between about 520 nm-600 nm. An especially preferred emission wavelength of about 540 nm (or higher) may be used, at which the most accurate readings for determining the total lipoprotein concentration (i.e. the concentration of HDL, IDL, LDL and VLDL, but also chylomicrons if present) may be observed.

The inventors have established that a number of discriminating dyes may be used in step (c) of the second aspect of the invention.

The inventors have been surprised to find that discriminating dyes exist that can discriminate between lipoproteins. It is most preferred that dye concentrations, excitation wavelengths and emission wavelengths are optimised for a particular dye. However it will be appreciate that the extent of optimisation, if any is required, will depend on the dye selected for use according to the invention.

Preferred discriminating dyes are able to bind selectively to HDL. Preferred dyes for binding selectively to HDL contain a fluorescent unit comprising a nitrogen atom linked to an aromatic structure and also connected to alkyl groups (i.e (alkyl)₂N(aromatic group). The alkyl group may be methyl or ethyl. The aromatic group preferably comprises at least two aromatic ring structures.

In one embodiment, step (c) comprises adding the dye Nile Red, or a functional analogue thereof, to the aliquot of the sample in order to assay for HDL in the sample. The formula of Nile Red is:

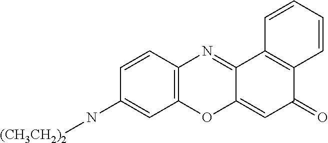

Preferably, in order to determine the HDL concentration in the sample using Nile Red, a calculation must be made of the excess fluorescence from Nile Red due to the presence of HDL. Firstly, the total lipoprotein concentration (measurement “A”) is measured by the linear correlation of lipophilic dye fluorescence with lipoprotein concentration (as determined by step (b)).

Secondly, Nile Red fluorescence is then calibrated with LDL (and/or VLDL as the fluorescence to concentration response must be essentially the same) at various concentrations to obtain a calibration curve with slope “X” and intercept “Y”. A skilled technician would know how to prepare a range of concentrations of LDL (and/or VLDL), and determine the respective fluorescence for each concentration.

Thirdly, an additional calibration curve is then constructed for a series of concentrations of HDL and a constant concentration of LDL to give slope “Z”. Fourthly, knowing the total lipoprotein concentration from the lipophilic dye measurement “A” and the excess Nile Red fluorescence of the unknown sample “B”, the concentration of HDL “C” in the unknown sample can be determined by the following equation:—

C=(B−(AX−Y))/Z

It will be appreciated that in the practice of the second aspect of the invention that pre-prepared or standard calibration curves may be used. Accordingly an operator need not repeat such calibrations (which are included herein for the sake of clarity) Furthermore devices developed to generate lipid profiles may have internal standards and/or have processing means that will allow for automatic calculation of HDL levels without user intervention.

Therefore, the method according to the second aspect of the invention may further comprise determining the concentration of HDL in the sample using fluorescence analysis. The method comprises steps (c) and (d) in which the discriminating dye such as Nile Red is added to a second aliquot of the sample. The dye binds to HDL and other lipoproteins but under appropriate excitation Nile Red fluoresces more and more strongly in proportion to the concentration of HDL in the sample. When this additional step is carried out, an even more detailed lipoprotein profile of the sample may be generated consisting of total lipoprotein concentration, and HDL concentration, which would be very useful to the clinician.

The inventors conducted a series of experiments to determine the optimum concentration of Nile Red, which should be added to the sample, to improve the accuracy of the determination of HDL in the sample, and this required considerable inventive endeavour. Accordingly, the concentration of the probe substance Nile Red added to the sample may be between approximately 0.1-1 mM. Advantageously, at this concentration of Nile Red, a more accurate determination of the concentration of the HDL concentration is possible.

Suitably, the concentration of Nile Red added to the sample may be between approximately 0.1-0.9 mM, more suitably, between approximately 0.2-0.8 mM (e.g. 0.2-0.7 mM), and even more suitably, between approximately 0.3-0.7 mM (e.g. 0.3-0.6 mM).). 4 mM Nile Red may be used although it is especially preferred to add Nile Red to the sample to a final concentration of about 0.6 mM.

The fluorescence of Nile Red is preferably induced by exciting the sample at an excitation wavelength of between about 400 nm-650 nm.

It is preferred that the excitation wavelength is 400 nm-650 nm; preferably, between about 420 nm-620 nm, more preferably, between about 500 nm-610 nm and even more preferably, between about 590 nm-610 nm. An excitation wavelength of about 600 nm may be used in connection with Nile Red which gives the largest discrimination (>5×) between the fluorescence response from Nile Red in HDL when compared with the other lipoproteins. When these excitation wavelengths are employed it is preferred that an agent is used that blocks the “fatty acid and drug binding domain” on Human Serum Albumin (HSA) as discussed in more detail below.

The resultant fluorescence from Nile Red may then be observed and measured at an emission wavelength of between about 540-700 nm, and more preferably, between about 570-650 nm. A preferred emission wavelength of about 620 nm may be used, at which the most accurate readings for determining the concentration of HDL may be observed.

The inventors investigated whether it was possible to further improve the accuracy of the individual assays used in the methods according to the first or second aspects of the invention, and so turned their attention to Human Serum Albumin (HSA), which is a major component of blood serum, having a concentration of approximately 30-50 mg/ml.

HSA is known to have at least two types of binding site that are capable of binding various ligands. A first type is referred to herein as “a hydrophobic domain” whereas a second type of domain is referred to herein as a “drug binding domains”. These domains are known to one skilled in the art and are distinguished from each other in a paper in Nature Structural Biology (V5 p 827 (1998)). This paper identifies the hydrophobic domain as one to which fatty acids may bind whereas the drug binding domain is capable of binding a number of drugs that may be associated with HSA.

From their experiments, the inventors have surprisingly established that dyes capable of fluorescing in the presence of lipoproteins may also bind to hydrophobic binding sites/domains of HSA. Hence, dyes used according to the invention may be ligands for HSA. In addition, surprisingly, the inventors found that the dyes (e.g. K-37 and Nile Red) fluoresce when bound to HSA. Therefore, while the inventors do not wish to be bound by any hypothesis, the inventors believe that this additional fluorescence, when bound to HSA, may cause a substantial background signal, which may distort and lead to significant errors in the determination of concentration of lipoprotein according to the first or second aspects of the invention.

As a result, the inventors investigated the effects of inhibiting the binding of dyes (e.g. K-37, and Nile Red) with HSA. In particular, they attempted to block the hydrophobic binding sites of HSA at which the probes K-37 and Nile Red bind and fluoresce. This work is described in Examples 4 and 5. While the inventors do not wish to be bound by any hypothesis, to their surprise, they found that inhibiting the binding of the dyes with the hydrophobic binding sites resulted in the fluorescence of the probe substance when bound to the lipoprotein molecules (HDL, LDL, VLDL) being a more accurate measure of the concentration of total lipoprotein in the sample than if no ligand binding inhibitor was added. The inventors also found that inhibiting binding of the ligand Nile Red to HSA improved the accuracy of the HDL determination.

Accordingly, it is preferred that the methods according to the invention comprises adding to the sample a ligand binding inhibitor that is adapted to substantially inhibit the binding of the dye substance to HSA, preferably, the hydrophobic binding sites thereof. It is especially preferred that the ligand binding inhibitor is also added to the sample prior to or at the same time as step (i) of the first aspect of the invention or steps (a) and/or (c) of the second aspect of the invention.

The ligand binding inhibitor may be hydrophobic. The inhibitor may be amphipathic. The ligand binding inhibitor may comprise a fatty acid or a functional derivative thereof, as well as other hydrophobic molecules. Examples of suitable derivatives of fatty acid, which may block the hydrophobic binding sites of HSA may comprise a fatty acid, its esters, acyl halide, carboxylic anhydride, or amide etc. A preferred fatty acid derivative is a fatty acid ester.

The fatty acid or derivative thereof may comprise a C₁-C₂₀ fatty acid or derivative thereof. It is preferred that the fatty acid or derivative thereof may comprise a C₃-C₁₈ fatty acid or derivative thereof, more preferably, a C₅-C₁₄ fatty acid or derivative thereof, and even more preferably, a C₇-C₉ fatty acid or derivative thereof.

It is especially preferred that the ligand binding inhibitor comprises octanoic acid (C₈) or a derivative thereof, for example, octanoate. Preferably, the ligand binding inhibitor is added as an alkali metal octanoate, preferably a Group I alkali metal octanoate, for example, sodium or potassium octanoate.

Preferably, between about 10-400 mM of the ligand binding inhibitor is added to the sample prior to carrying out an assay according to the first or second aspects of the invention, more preferably, between about 20-200 mM, and even more preferably, between about 50-150 mM is added. It is especially preferred that about 100 mM of the inhibitor is added. Hence, in a preferred embodiment of the method, about 100 mM of sodium octanoate may be added to the sample before or at the same time as carrying out step (i) of the first aspect of the invention or steps (a) and (c) of the second aspect of the invention.

In a preferred embodiment of the first aspect of the invention, a ligand binding inhibitor, for example, about 100 mM sodium octanoate, is first added to an aliquot taken from the sample, with approximately 0.4 mM DPH or 0.5 mm DPO, prior to carrying out the fluorescence measurement of the total lipoprotein concentration in step (i) of the method.

In a preferred embodiment of the second aspect of the invention, a ligand binding inhibitor, for example, about 100 mM sodium octanoate, is first added to a first aliquot of the sample, with approximately 0.4 mM DPH (step (a)) and also about 100 mM sodium octanoate, added to a second aliquot, with approximately 0.4 mM or more preferably 0.6 mM of the Nile Red probe, prior to carrying out the fluorescence measurement of the HDL concentration in the method (step (c)).

In another preferred embodiment of the second aspect of the invention, a ligand binding inhibitor, for example, about 100 mM sodium octanoate, is first added to a first aliquot of the sample, with approximately 0.5 mM DPO (step (a)) and also about 100 mM sodium octanoate, added to a second aliquot, with approximately 0.1 mM of Nile Red, prior to carrying out the fluorescence measurement of the HDL concentration in the method (step (c)).

The inventors have additionally found that Nile Red also interacts with the drug binding domain on HSA that is referred to above. Ligands for this drug binding domain include drug molecules such as: thyroxine, ibuprofen, diazepam, steroid hormones and their derivatives (drugs), haem, bilirubin, lipophilic prodrugs, warfarin, coumarin based drugs, anaesthetics, diazepam, ibuprofen and antidepressants (e.g. thioxanthine). The inventors have found that agents may be used to block this drug binding domain and that this results in further improvement of assay results with Nile Red. The abovementioned drugs, or any other molecule with affinity to this domain, may be used as agents for blocking the drug binding domain of HSA. However it is most preferred that benzoic acid or a derivative thereof (e.g. trichlorobenzoic acid or triiodobenzoic acid) is used to block the drug binding domain.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

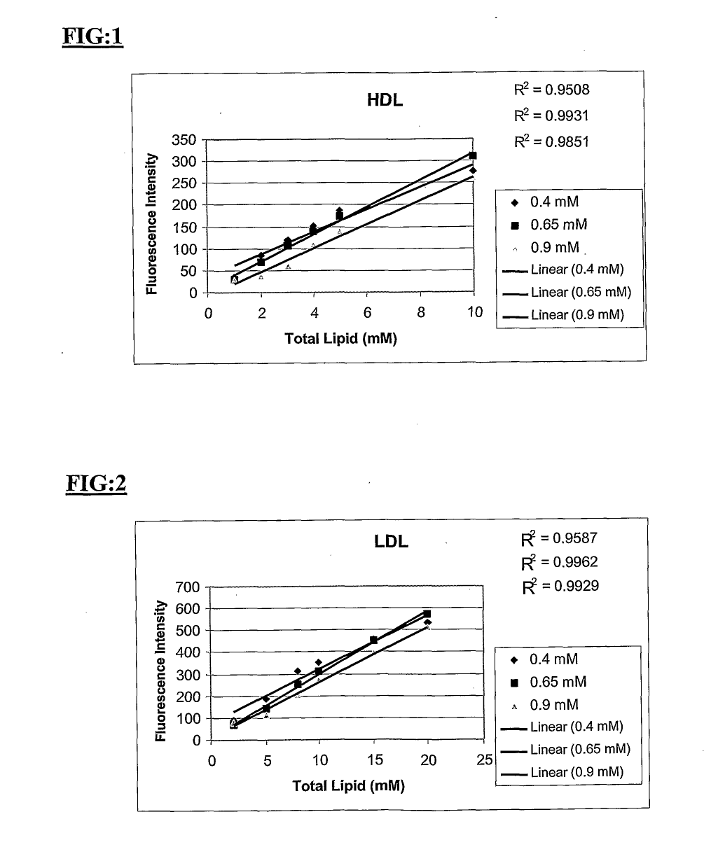

FIG. 1 is a graph showing fluorescence intensity against total lipid concentration for K-37 at three concentrations (0.4 mM, 0.65 mM and 0.9 mM) in HDL as referred to in Example 1;

FIG. 2 is a graph showing fluorescence intensity against total lipid concentration for K-37 at three concentrations (0.4 mM, 0.65 mM and 0.9 mM) in LDL as referred to in Example 1;

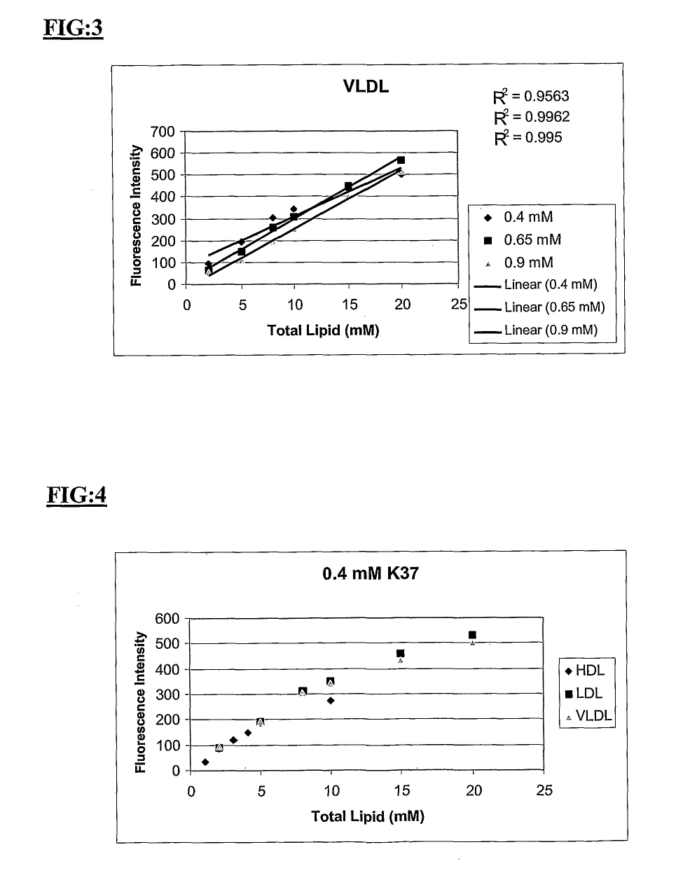

FIG. 3 is a graph showing fluorescence intensity against total lipid concentration for K-37 at three concentrations (0.4 mM, 0.65 mM and 0.9 mM) in VLDL as referred to in Example 1;

FIG. 4 is a graph showing fluorescence intensity against total lipid concentration for 0.4 mM K-37 in HDL, LDL, and VLDL as referred to in Example 1;

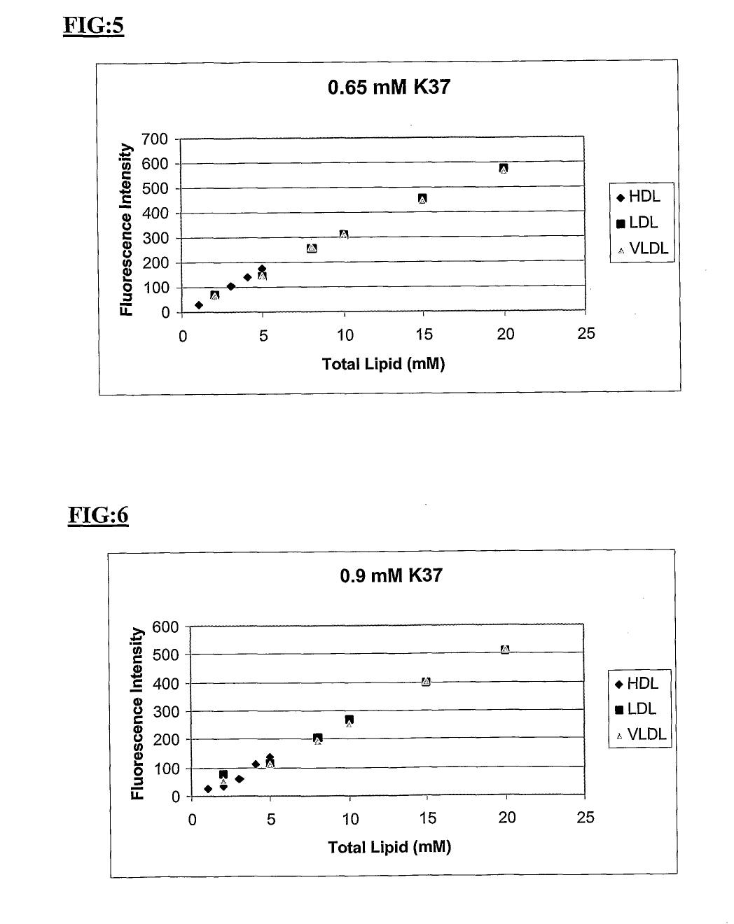

FIG. 5 is a graph showing fluorescence intensity against total lipid concentration for 0.65 mM K-37 in HDL, LDL, and VLDL as referred to in Example 1;

FIG. 6 is a graph showing fluorescence intensity against total lipid concentration for 0.9 mM K-37 in HDL, LDL, and VLDL as referred to in Example 1;

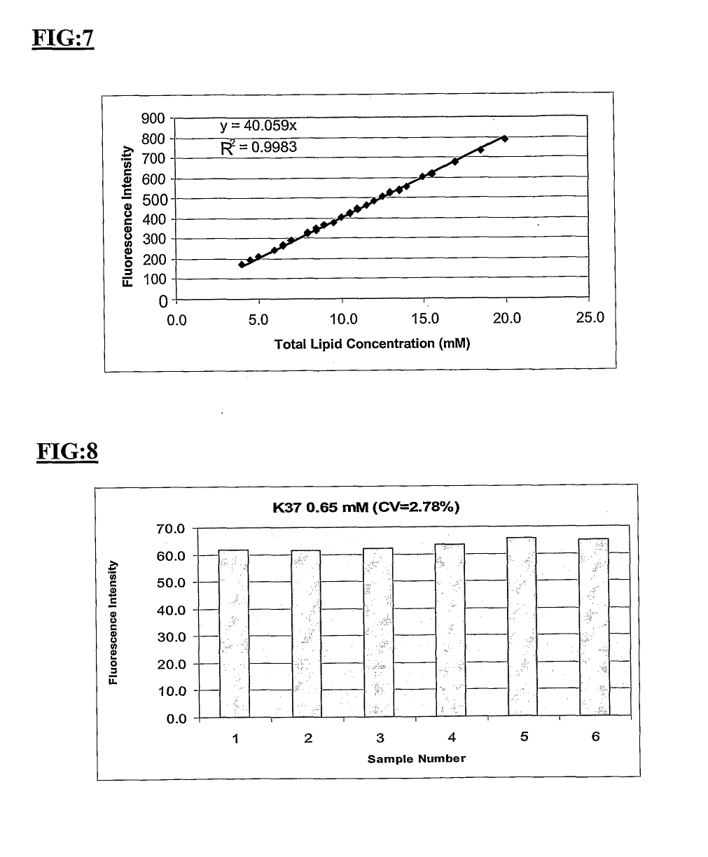

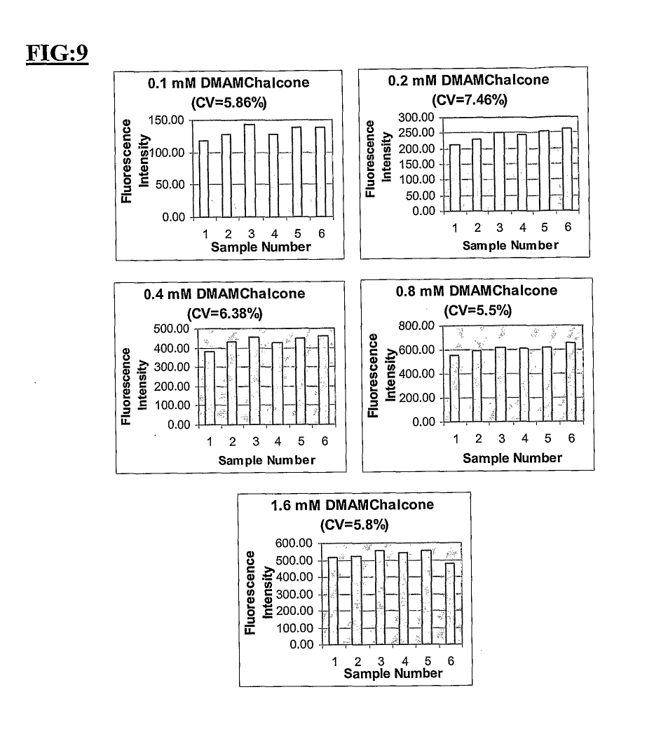

FIG. 7 is a graph showing fluorescence intensity against total lipid concentration for 0.65 mM K-37 in a series of HDL, LDL, and VLDL mixtures as referred to in Example 1;

FIG. 8 is a graph illustrating fluorescence intensity against the 6 sample solutions of Example 2 for 0.65 mM K-37;

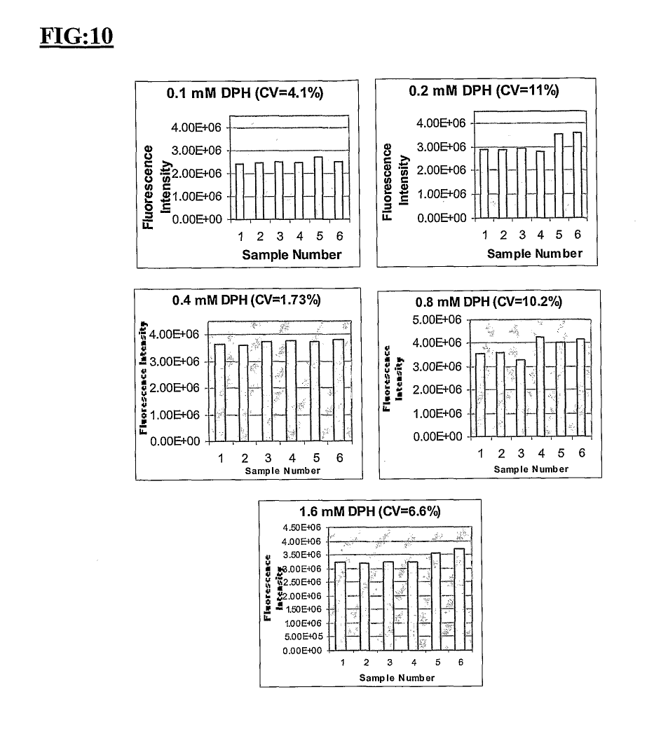

FIG. 9 provides graphs illustrating fluorescence intensity against the 6 sample solutions of Example 2 for a range of concentrations of 4-Dimethylamino methylchalcone;

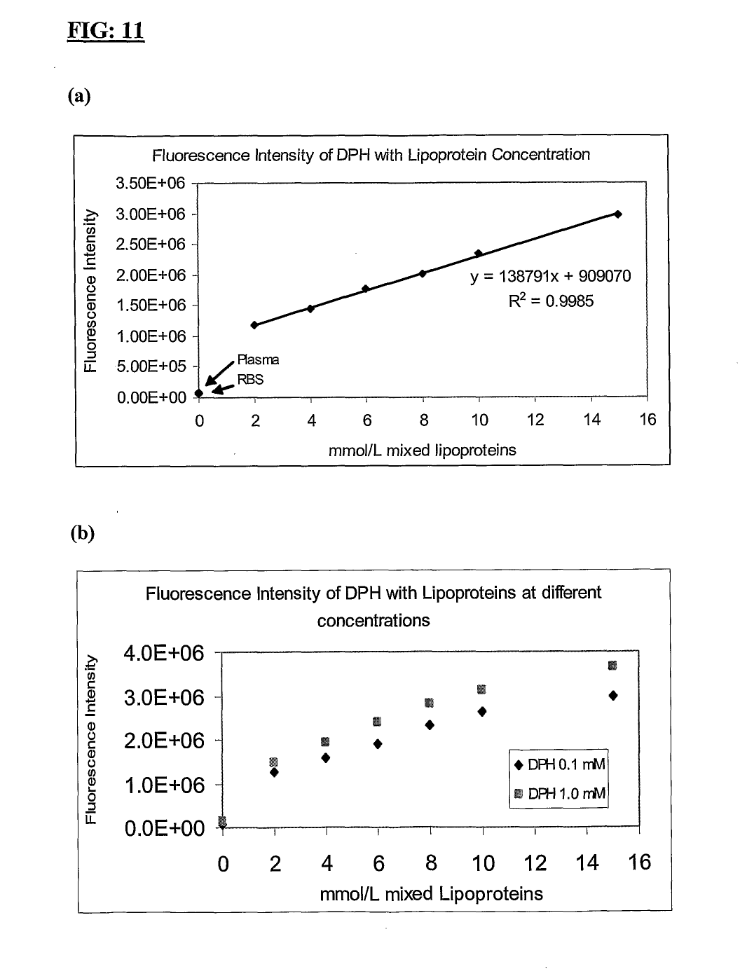

FIG. 10 provides graphs illustrating fluorescence intensity against the 6 sample solutions of Example 2 for a range of concentrations of diphenylhexatriene (DPH);

FIG. 11 provides graphs illustrating (a) fluorescence intensity of 0.5 mM DPH over a range of concentrations of mixed lipoproteins as described in Example 2; and (b) fluorescence intensity of 0.1 mM, 1.0 mM and 4.0 mM DPH over a range of concentrations of mixed lipoproteins as described in Example 2

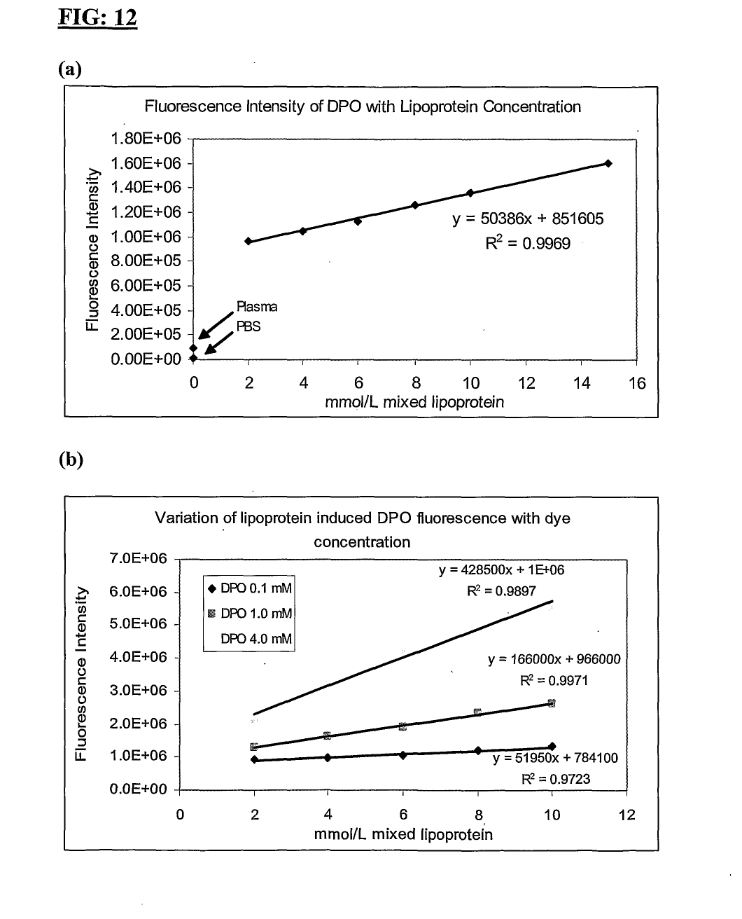

FIG. 12 provides graphs illustrating (a) fluorescence intensity of 0.5 mM DPO over a range of concentrations of mixed lipoproteins as described in Example 2; and (b) fluorescence intensity of 0.1 mM and 1.0 mM DPO over a range of concentrations of mixed lipoproteins as described in Example 2;

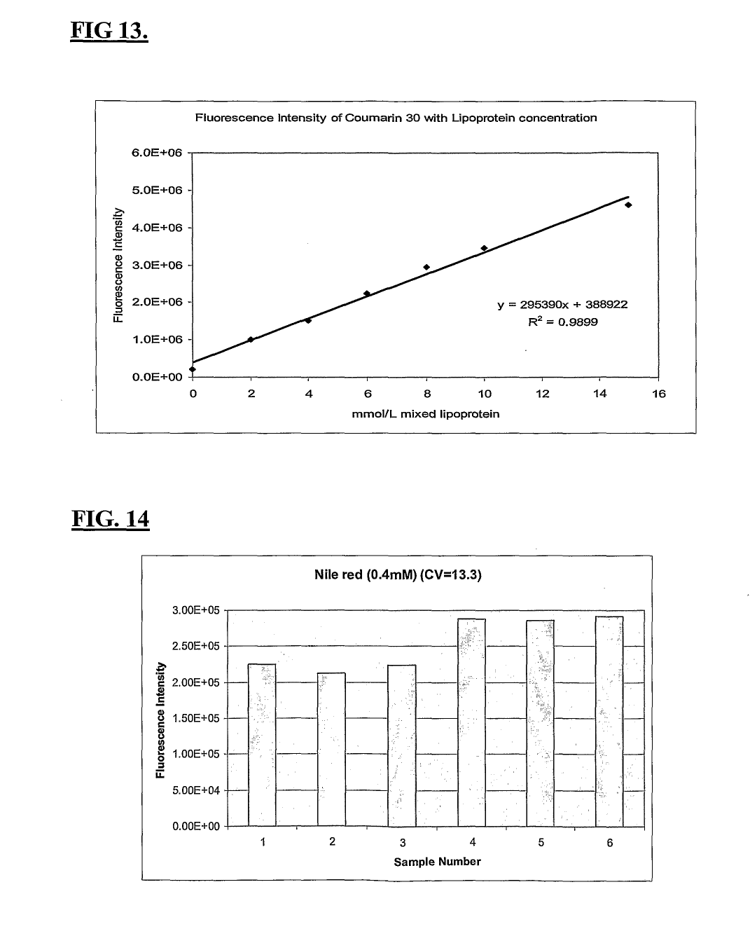

FIG. 13 is a graph illustrating fluorescence intensity of Coumarin 30 over a range of concentrations of mixed lipoproteins as described in Example 2;

FIG. 14 is a graph illustrating fluorescence intensity against the 6 sample solutions of Example 3 for 0.4 mM Nile Red;

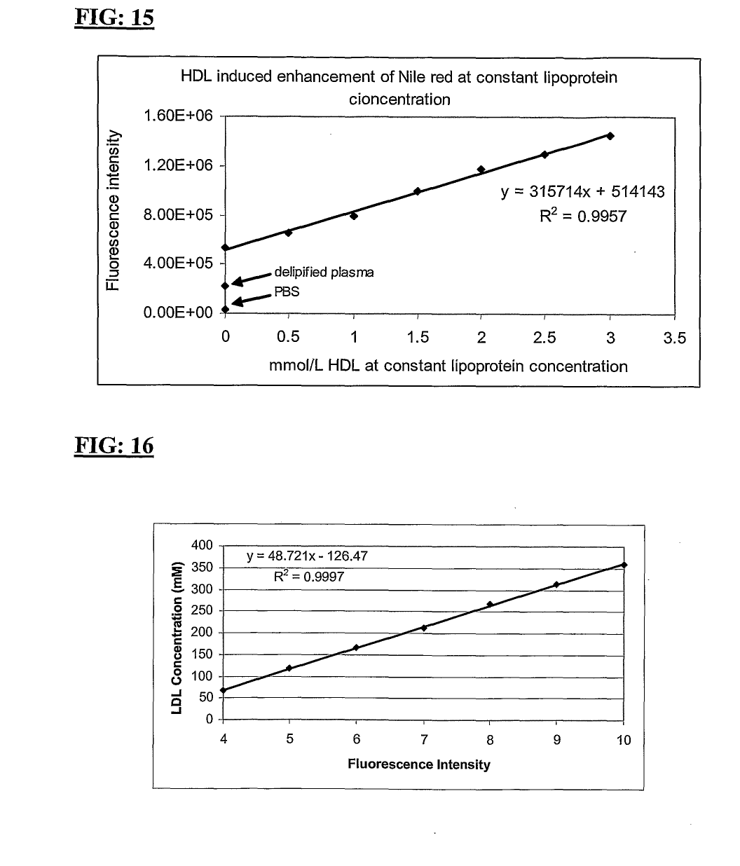

FIG. 15 is a graph illustrating Nile Red fluorescence against HDL concentration as referred to in Example 3;

FIG. 16 is a calibration curve of LDL concentration against fluorescence intensity as referred to in Example 5;

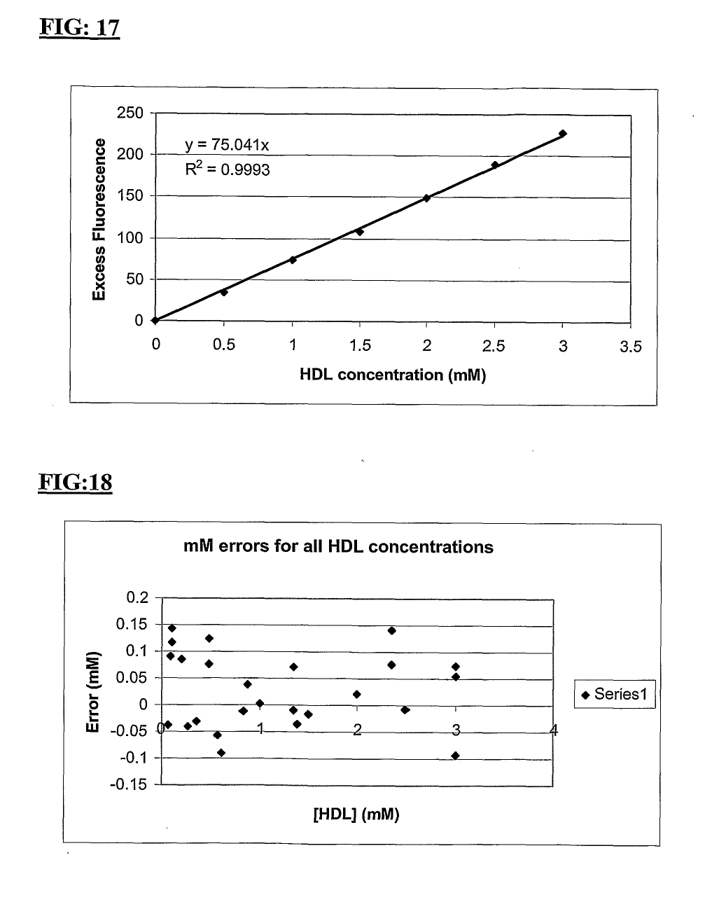

FIG. 17 is a calibration curve of excess fluorescence against HDL concentration as referred to in Example 5;

FIG. 18 is a graph showing errors against HDL concentration as referred to in Example 5;

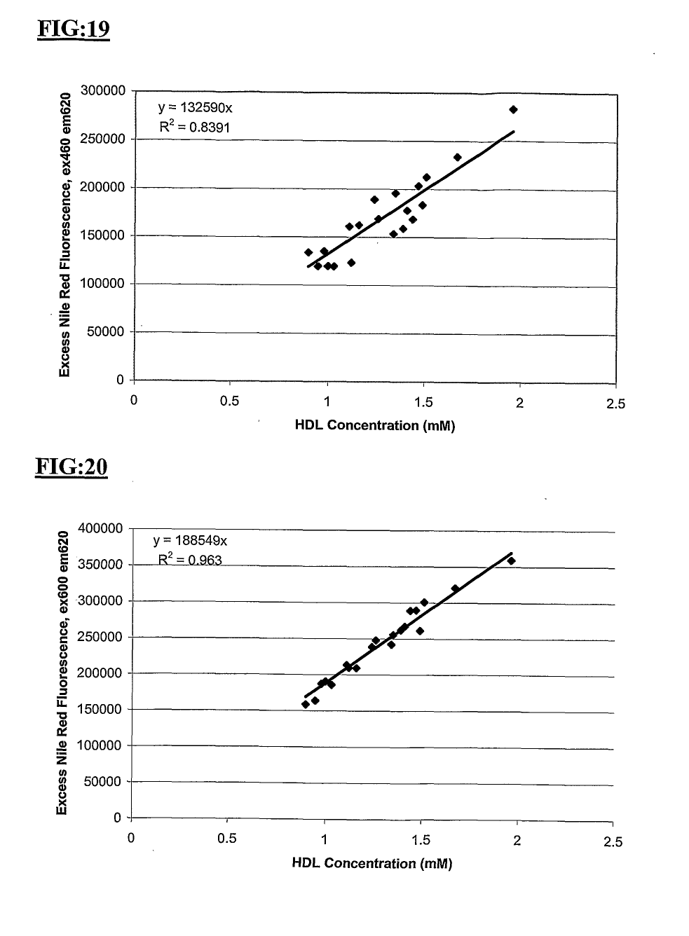

FIG. 19 is a graph illustrating Nile Red fluorescence (ex460 nnm and em620 nm) against HDL concentration as referred to in Example 5;

FIG. 20 is a graph illustrating Nile Red fluorescence (ex600 nnm and em620 nm) against HDL concentration as referred to in Example 5; and

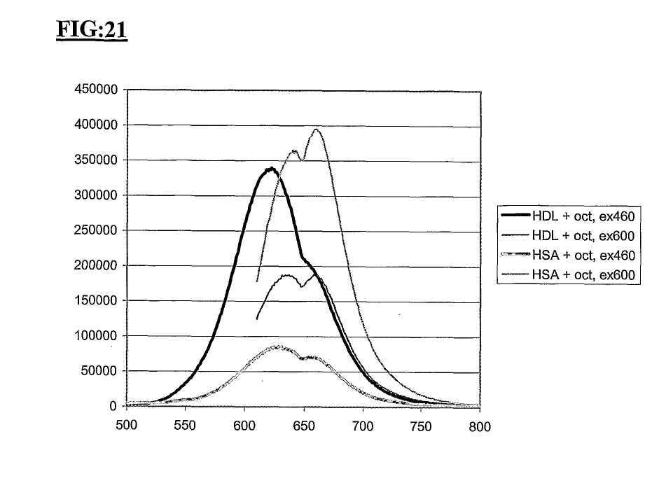

FIG. 21 is a graph illustrating a spectral analysis of Nile Red fluorescene in the presence of HDL (+octanoate) or HSA (+octanoate) at excitation wavelengths of 460 nm and 600 nm.

### EXAMPLE 1

The inventors carried out a series of experiments in order to investigate whether or not fluorescent dyes may be used to determine the concentration of total lipoproteins according to the first aspect of the invention

The inventors noted that WO 01/53829A1 discloses that K-37, at concentrations around 0.1 mM, is known to have different fluorescence intensity responses to lipoprotein classes. This is because the dye-lipoprotein complexes have different fluorescence lifetimes therefore different quantum yields.

The inventors decided to investigate whether or not any assay conditions might exist that would mean it was possible to use K-37 to detect the concentration of total lipoproteins (which equates to total triglycerides plus cholesterol plus cholesterol esters as it is assumed that all lipids are bound to lipoproteins) in samples by means of a simple fluorescence assay.

### 1.1. Methods

The dye, K-37, which was dissolved in dimethyl formamide (DMF), was added at a range of different concentrations, to a concentration series of HDL, LDL, and VLDL dissolved in phosphate buffered saline. The objective of the experiment was to obtain a linear and equal relationship between fluorescence and lipoprotein concentration for each particle type (HDL, LDL, and VLDL), across the range of lipoprotein concentrations that would be encountered in real plasma or serum samples. Fluorescence intensity was measured in a Perkin-Elmer LS50 fluorimeter, at an excitation wavelength of 450 nm, and at an emission wavelength of 540 nm.

### 1.2 Results

FIGS. 1 to 3 illustrate the fluorescence intensity versus total lipoprotein concentration for K-37 at three concentrations, i.e. 0.4 mM, 0.65 mM, and 0.9 mM, in HDL, LDL, and VLDL in phosphate buffered saline. The R² values are shown for linear fits to each series (0.4 mM at the top, 0.65 mM in the centre, and 0.9 mM below). The same data are also plotted in FIGS. 4 to 6, and are grouped by K-37 concentration.

The conclusions from these experiments were are that:—

1) For all three lipoprotein particle types (HDL, LDL, and VLDL), the R² shows that there is a good linear relationship between total lipoprotein concentration and fluorescence intensity at a K-37 concentration of 0.65 mM. Good linear relationships are also observed for 0.9 mM K-37 in LDL and VLDL, but the linearity at 0.9 mM K-37 in HDL is a little poorer. Linearity is poorer for all lipoproteins with 0.4 mM K-37. It is noteworthy that while it still works at concentrations where linearity is poorer, it is less accurate. However, non-linearities may be dealt with using polynomial fitting.
2) Two factors are thought to affect linearity. At a low dye concentration, there is a flattening off of the response at high total lipoprotein concentration. While the inventors do not wish to be bound by any hypothesis, they believe that this occurs because there is insufficient dye available to fully occupy the lipoprotein particles. At high dye concentrations, there is a flat response with low total lipoprotein concentration. This is caused by self-quenching of the fluorescence when the dyes are packed very closely in the particles.
3) A K-37 concentration of 0.65 mM gives linear and very similar fluorescence responses for all the lipoprotein particle types across the appropriate range when measured in phosphate buffered saline.

Accordingly, 0.65 mM K-37 was then added to a series of HDL/LDL/VLDL mixtures, and fluorescence intensity was measured as described above. The data is illustrated in FIG. 7. As can be seen, the total lipoprotein concentration is highly correlated with fluorescence intensity (R²=0.9983), confirming that this concentration of K-37 (0.65 mM) is suitable for highly accurate measurements of total lipoprotein concentration. When applying this to biological samples from patients, the inventers observed some curvature at high lipid concentration. Consequently a concentration of 0.7 mM K-37 was chosen as the optimal K-37 concentration for use in serum or plasma. Hence, this concentration was selected as the most suitable concentration for the method according to the invention.

### EXAMPLE 2

The data presented in Example 1 made the inventors realise that dyes with similar properties to that of K-37 may be used in assays according to the first aspect of the invention. Further experiments are described in Example 2 which illustrate a whole class of lipophilic dyes (as defined in the first aspect of the invention) may be employed in the fluorescent measurement of the total lipoprotein content of a sample.

### 2.1 Methods

The methods employed in Example 1 were adapted to test a number of different dyes.

The dyes were tested by applying a range of concentrations of dye (at final concentrations of 0.1, 0.2, 0.4, 0.8, 1.6 mM) to each of six solutions containing different ratios of lipoprotein classes (all with a final total lipoproteinconcentration of 6.0 mmole/L) as illustrated in Table 1. The fluorescence intensity of the solutions were then measured as previously described.

TABLE 1

| Solution | | | |
|---|---|---|---|
| Number | HDL | LDL | VLDL |
| 1 | 1 mM | 0.5 mM | 4.5 mM |
| 2 | 1 mM | 2.5 mM | 2.5 mM |
| 3 | 1 mM | 4.5 mM | 0.5 mM |
| 4 | 3 mM | 0.5 mM | 2.5 mM |
| 5 | 3 mM | 1.5 mM | 1.5 mM |
| 6 | 3 mM | 2.5 mM | 0.5 mM |

Dyes considered to be useful according to the first aspect of the invention were assessed by calculating the coefficient of variances (CV) of the fluorescence intensity of a dye over the six solutions of different lipoprotein ratios for a given dye concentration. The CV is defined as the standard deviation of the six fluorescence intensity measurements divided by the mean of the measurements and multiplied by 100 to give a percentage value. Thus a low value (<10%) was taken to suggest that the dye was non-discriminating between the different classes of lipoprotein in the solution and was therefore useful according to the first aspect of the invention. Dyes with a value of 3% or less represented most preferred dyes for use according to the first aspect of the invention.

### 2.2 Results

The inventors established that many dyes were unsuitable for use according to the method of the first aspect of the invention. Many dyes were incapable of binding to lipoproteins and fluorescing. However of those that would fluoresce, many would either:
(a) not fluoresce such that there was consistent reading between sample 1-6 and had CV values >10%; or
(b) if resulted in CV values of <10%, needed exciting or emitted at a wavelength that would be impaired by fluorescence from biological samples such as serum or plasma.

Therefore the inventors dismissed a number of dyes as being suitable for use according to the first aspect of the invention. For instance, experiments were conducted with the dye pyrene. This dye was capable of binding to at least some lipoproteins and fluoresced when bound thereto (data not shown). However it had an excitation maximum at 320 nm and would therefore have been unsuitable for use with biological samples (many molecules, and not just lipoproteins, in a biological sample would also fluoresce if a sample was excited at this wavelength).

However amongst the dyes tested the inventors were surprised to find that lipophilic dyes, and in particular lipophilic dyes with two phenolic groups were just as good as K-37 at its optimised concentration for measuring the total content of a lipoprotein in a sample.

Dyes according to the first aspect of the invention included:

### 2.2.1 Chalcone Based Dyes

0.65 mM K-37 resulted in CV of 2.78% (see FIG. 8). This indicates that at a concentration of 0.65 mM the dye does not discriminate between lipoprotein particles and confirms its usefulness according to the first aspect of the invention.

FIG. 9 illustrates the experimental findings for 4-Dimethylamino methylchalcone (DMAMC). This dye had CVs well below 10%. The excitation maximum for this dye is 420 nm and the emission maximum is 490 nm. These wavelengths are suitable for a blood plasma assay and this dye represents a preferred lipophillic dye for use according to the first aspect of the invention.

2.2.2 Ph-[C—C═C]ₙ—C-Ph Dyes and Derivatives Thereof.

FIG. 10 illustrates the experimental results for DPH. It did not discriminate between the lipoprotein classes and gave a best CV (1.7%) at concentration of 0.4 mM dye. This illustrates that DPH is useful according to the first aspect of the invention.

DPH has an excitation maximum at 350 nm and emission maximum at 440 nm. However the inventors found it to be excitable to about 400 nm. This avoided much of the contaminating fluorescence background associated with blood plasma at around 359 nm (and below) and made the dye useful according to the first aspect of the invention.

DPH represents a preferred dye for use according to the first aspect of the invention. A skilled person will appreciate that DPH is a basic fluorophoric group found within a number of lipophilic dye. Various ring substitutions may be made to the DPH molecule to adjust the fluorescence properties of the dye. Such dyes may also be used for measuring total liproprotein according to the first aspect of the invention.

FIG. 11 (a) illustrates fluorescence intensity of 0.5 mM DPH over a range of concentrations of mixed lipoproteins; and (b) fluorescence intensity of 0.1 mM, 1.0 mM and 4.0 mM DPH over a range of concentrations of mixed lipoproteins. These graphs further illustrate the linear relationship between fluorescence and lipoprotein concentration and thereby demonstrate the suitability of DPH for use according to the first aspect of the invention.

FIG. 12 (a) and (B) present similar data for DPO, another preferred dye for use according to the first aspect of the invention, and also demonstrate the suitability of DPO for use according to the first aspect of the invention.

### 2.2.3 Coumarin Dyes

FIG. 13 is a graph illustrating fluorescence intensity of Coumarin 30 over a range of concentrations of mixed lipoproteins. The graph illustrates the linear relationship between fluorescence and lipoprotein concentration and thereby demonstrates the suitability of Coumarin 30 for use according to the first aspect of the invention.

### 23 Conclusions

Taken together these results illustrate that lipophilc dyes, and particularly the biphenolic dyes discussed above, are useful dyes for assaying total lipoprotein content of a sample according to the first aspect of the invention.

### EXAMPLE 3

The series of experiments conducted to generate the data for Example 2 also lead the inventors to realise that some dyes can discriminate between lipoprotein classes and may therefore be used in step (c) of the method according to the second aspect of the invention.

### 3.1 Methods

The methods employed in Example 2 were repeated.

### 3.2 Results

### Nile Red

HDL induced fluorescence enhancement of Nile red when excited at 460 nm and the emission monitored at 620 nm. At this excitation the fluorescence enhancement in HDL is twice that of both VLDL and LDL. However the CV of the first 3 samples came to 2.78 and 1.0% for samples 4 to 6 indicating that the dye does not distinguish between LDL and VLDL. The fluorescence enhancement when Nile red was excited at 600 nm is >5 times stronger for HDL.

FIG. 14 illustrates how 0.4 mM Nile Red discriminated between lipoprotein classes.

FIG. 15 shows that Nile red is virtually non-fluorescent in PBS, is only moderately fluorescent in plasma due to protein binding and further increases, in fluorescence intensity when 6 mmol/L lipoprotein is added. There is a strong increase in the fluorescence intensity of Nile red as the HDL content is increased at constant lipoprotein concentration demonstrating that Nile red discriminates for HDL. The inventors believe that this fluorescence enhancement arises from binding of the dye to the protein lipid interface of the HDL particles. HDL is more than 50% protein, mainly ApoA which winds its way through the particle quite differently to VLDL, IDL and LDL which possess copies of a surface bound protein ApoB making up far less of the particles by weight and providing less of the protein/lipid interface.

These preliminary experiments were expanded (see Example 5) to confirm that Nile Red is a useful discriminating dye that may be used in the method according to the second aspect of the invention.

### EXAMPLE 4

The inventors conducted further investigations to optimise the methods according to the invention. To this end, they realised that HSA possesses a hydrophobic binding sites in which dyes binds and fluoresces. This additional fluorescence when bound to HSA can cause a substantial background signal, which distorts and thereby causes significant errors in the measurement of the lipoprotein molecules, i.e. HDL, LDL and VLDL. They therefore decided to investigate if they could block the hydrophobic binding sites in HSA with a ligand binding inhibitor, such as sodium octanoate, to see if the additional fluorescence could be minimised. It was envisaged that inhibiting the binding of dye with HSA in this way would improve the accuracy of the results obtained using dye fluorescence measurements.

Experiments, for illustrative purposes, were conducted using K-37. However a skilled person will understand that the data presented in this Example will be applicable to any of the lipophilic dyes and discriminating dyes according to the invention.

### 4.1 Methods

The dye K-37 was added at a concentration of 0.5 mM to LDL at a total lipid concentration of 5 mM, in the presence and absence of 50 mg/ml HSA. Measurements were made with and without the addition of 0.1 M sodium octanoate, which acted as a ligand binding inhibitor.

### 4.2 Results

Fluorescence intensity was measured for all samples and is summarised in Table 2.

TABLE 2

| Sample | Fluorescence Intensity |
|---|---|
| K-37 plus 5 mM LDL | 213500 |
| K-37 plus 50 mg/ml HSA | 79300 |
| K-37 plus 5 mM LDL + octanoate | 209700 |
| K-37 plus 50 mg/ml HSA + octanoate | 3600 |

The results show that the fluorescence intensity of K-37 in LDL alone is 213500 units. The fluorescence intensity of K-37 when octanoate is added to LDL is 209700 units (i.e. about the same as without octanoate), which suggests that the presence of octanoate does not contribute to the fluorescence intensity of K-37 bound to LDL by itself. The fluorescence intensity of K-37 bound to HSA is 79300 units, whereas that of K-37 in the present of HSA and octanoate is 3600. This illustrates that HSA contributes to K-37 fluorescence and is therefore an interfering signal. The addition of octanoate significantly reduces this interference and thereby obviates the disruptive effects of HSA. The results therefore show a large suppression of fluorescence intensity for K-37 with HSA in the presence of octanoate, but little effect on K-37 fluorescence in LDL. This showed that octanoate is remarkably successful at blocking the K-37 binding site on HSA, making the K-37 fluorescence a true measure of total lipoprotein concentration.

### 4.3 Conclusions

Accordingly, the inventors believe that a ligand binding inhibitor such as octanoate, which binds the hydrophobic binding sites of HSA, can be added to the blood sample prior to measuring lipophilic dye fluorescence to improve the accuracy of the total lipoprotein concentration. In addition, the inventors suggest that this technique can also be used to block the binding of other ligands to the hydrophobic binding sites of HSA, and to displace ligands that may be already bound thereto, and which have a lower affinity for HSA than the octanoate.

### EXAMPLE 5

The inventors expanded the experiments described in Example 3 to confirm that discriminating dyes may be used to distinguish between the different types of lipoprotein present in a blood sample.

For illustrative purposes the inventors chose to use Nile Red as an example of a discriminating dye that may be used according to the second aspect of the invention.

### 5.1 Methods

The principle of the measurement is that the probe Nile Red is more fluorescent in HDL than in LDL, and VLDL, the latter having very similar but not identical fluorescence responses with concentration. The measurement is more complicated than measurement for total lipoprotein (according to the first aspect of the invention), as a calculation must be made of excess fluorescence from Nile Red in HDL, and not simply total fluorescence of all lipoproteins. The procedure is as follows:—

### 5.1.1 Calibration

0.5 mM Nile Red dissolved in dimethylformamide was mixed with LDL at varying total lipoprotein concentrations usually between 4 and 10 mM (typically 50 microlitres of dye are mixed with 50 microlitres of lipoprotein and 1 ml of phosphate buffered saline). Samples were put in a spectrofluorimeter and fluorescence intensity was measured (excitation wavelength 450 nm, emission wavelength 600 nm). Fluorescence intensity was plotted against LDL total lipid concentration, giving a straight calibration line with slope “X” and intercept “Y”, as shown in FIG. 16.

The procedure was then repeated for mixtures of LDL and HDL. HDL was added at concentrations of between 0 and 3.0 mM, with LDL added to keep the total lipoprotein concentration at 6 mM for all samples (but 3-12 mM would be the limits). Fluorescence intensities for these samples were then measured. A plot was then made of excess fluorescence due to the presence of HDL, giving a straight calibration line having slope “Z”, as illustrated in FIG. 17.

### 5.1.2 Measurements of Unknowns

0.5 mM Nile Red dissolved in dimethylformamide was mixed with the sample under investigation. The sample was put into a fluorimeter and fluorescence intensity was measured under the same conditions as for the calibration described above.

5.1.3 Calculation of HDL Concentration

Calculation of HDL requires knowledge of the total lipoprotein concentration “A”, which can for example but not exclusively be measured from the fluorescence intensity of a lipophilic dye used in the method according to the first aspect of the invention. For a particular sample, the fluorescence intensity that would be expected if the sample contained no HDL is obtained from the calibration line shown in FIG. 17. The measured fluorescence intensity minus this calculated fluorescence intensity is the excess fluorescence due to HDL present in the sample.

The HDL concentration “C” in the unknown sample can then be obtained using the calibration line shown in FIG. 17 and the following equation:—

C=(B−(AX−Y))/Z

A range of concentrations of HDL/LDL/VLDL mixtures were prepared intended to cover the range of concentrations that would be expected in real clinical samples. The calibration data discussed above were used to calculate HDL concentrations from the mixtures. FIG. 18 illustrates errors between actual HDL concentration and HDL concentration determined from Nile Red fluorescence, showing a maximum error of only approximately 0.15 mM. The inventors further refined the concentration of Nile Red for use in samples of serum to be 0.4 mM.

As a result of these data, the inventors have shown that it is possible to distinguish between the types of lipoprotein present in a sample, and to determine the concentration of HDL using the dye Nile Red.

5.1.4 Use of Nile Red in Conjunction with a HSA Blocker

Following the findings described in Example 4, concerning the addition of octanoate to block the hydrophobic binding sites of HSA, the inventors then observed that Nile Red also binds to HSA and fluoresces. This additional fluorescence of Nile Red when bound to HSA also causes a substantial background signal, which distorts and thereby causes significant errors in the measurement of HDL. They therefore decided to block the hydrophobic binding sites in HSA with the same ligand binding inhibitor as for K-37 blocking, i.e. sodium octanoate. The experiments conducted with Nile Red and HSA, were based on those discussed in Example 4, and all using 0.5 mM Nile Red.

TABLE 3

| Sample | Fluorescence Intensity |
|---|---|
| Nile Red plus 5 mM LDL | 187.532 |
| Nile Red plus 50 mg/ml HSA. | 58.905 |
| Nile Red plus 5 mM LDL + 50 mM | 183.786 |
| octanoate | |
| Nile Red plus 50 mg/ml HSA. + 50 mM | 9.118 |
| octanoate | |
| PBS + 50 mM Octanoate | 7.382 |

The results presented in Table 2 show that the fluorescence intensity of Nile Red in LDL alone is 187.532 units. The fluorescence intensity of Nile Red when octanoate is added to LDL is 183.786 units (i.e. about the same as without octanoate), which suggests that the presence of octanoate does not contribute to the fluorescence intensity of dye bound to LDL by itself. The fluorescence intensity of Nile Red bound to HSA is 58.905 units, whereas that of Nile Red in the present of HSA and octanoate is 9.118. This illustrates that HSA contributes to Nile Red fluorescence and is therefore an interfering signal. The addition of octanoate significantly reduces this interference and thereby obviates the disruptive effects of HSA. The results therefore show a large suppression of fluorescence intensity for Nile Red with HSA in the presence of octanoate, but little effect on Nile Red fluorescence in LDL.

This showed that octanoate is remarkably successful at blocking the Nile Red binding site on HSA, making the Nile Red fluorescence a true measure of lipoprotein concentration. Accordingly, the inventors believe that a ligand binding inhibitor such as octanoate, which can fit in the hydrophobic binding sites of HSA, can be added to the blood sample prior to measuring the fluorescence of Nile Red to improve the accuracy of the lipoprotein (HDL) concentration. Subsequent to this work the inventors found that 0.4 mM Nile Red and 50 mM, or more preferably about 100 mM, octanoate were optimal for the analysis of serum samples.

### 5.1.5 Further Optimisation of Assays Utilizing Nile Red

Further tests were performed on human serum samples to investigate optimum excitation wavelengths for inducing fluorescence indicative of HDL levels according to the method of the invention.

The inventors tested a number of wavelengths and have established that, when using Nile Red, that an excitation wavelength of 600 nm and an emission wavelength of 620 nm gives optimal results (see FIG. 19). The inventors were surprised that this excitation wavelength was optimal because it is to the very long wavelength edge of the spectrum.

For certain samples the inventors observed a noisier plot with an excitation wavelength of 460 nm and an emission wavelength of 620 nm (see FIG. 20).

The inventors believe that Nile Red is about 5 times more fluorescent in HDL than VLDL and LDL when excited at 600 nm as opposed to excitation at 460 nm where it is only about 2 times more fluorescent. This gives a better signal to noise when subtracting from the standard curve of LDL plus VLDL.

The inventors have found that the optimal concentration of Nile red is around 0.6 mM.

Although the inventors do not wish to be bound by any hypothesis, they believe the “noise” observed in serum samples, excited at 460 nm, is an effect of signal-to-noise. The inventors have noted that Nile Red binds to HSA and particularly at low lipid concentrations. They therefore performed a spectral analysis of Nile Red fluorescene in the presence of HDL (+octanoate) or HSA (+octanoate) both at an excitation wavelength of 460 nm and 600 nm (see FIG. 21). These experiments resulted in unexpected spectral behaviour which the inventors believe may be explained by the fact that Nile red is in a rigid but polar environment (binding site on HSA) and the Nile red exhibits twisted intramolecular charge transfer (TICT) (Journal of Photochem and Photobiol A:Chemistry 93 (1996) 57-64) that shifts the excitation and emission to longer wavelengths. The molecule in this excited state has a different dipole moment and so behaves like a different species. In exciting at 600 nm the better signal-to-noise due to the larger difference in signal between Nile Red in HDL compared with other lipoproteins more than compensates for the excitation of the TICT state because TICT fluorescence is excluded by the 620 nm emission wavelength setting. In other words, while the HSA/NileRed was excited more optimally at 600 nm its fluorescence is rejected by the instrument.

This led the inventors to realise that the HDL/Nile red assay may be improved further by using an additional blocker. They tried agents that block the drug binding domain of HSA. To their surprise they found that agents such as benzoic acid, and its trichoro and triiodo derivatives, all worked to displace the Nile Red from HSA without affecting the lipoprotein fluorescence at about 5 mM. The benzoic acid has the added bonus of quenching the Nile Red residual fluorescence in solution by about 20%.

### EXAMPLE 6

Examples 1 and 2 illustrate how fluorescence measurements of the lipophilic dyes may be used to determine the concentration of total lipoproteins in a sample whereas Examples 3 and 5 illustrate how the fluorescence measurements of discriminating dyes may be used to determine the concentration of HDL in a sample.

In view of these results, the inventors realised that it is possible to create a single parallel method for analysing the lipid composition of a patient's blood sample in order to create a lipoprotein profile for that patient. This method represents the second aspect of the invention and consists of two assays, both of which can be carried out under very similar conditions, and hence, can produce results very quickly. A preferred method according to the second aspect of the invention is provided below.

### Method

A blood sample is initially taken from a patient, and then centrifuged using well-established conventional techniques, in order to separate the serum. The serum is then separated in to two 1 ml aliquots (a, & b), each of which is subjected to biochemical analysis to determine the concentration of a lipid component. Aliquot (a) is used to determine the concentration of total lipoprotein; and aliquot (b) is used to determine the concentration of HDL, as described below.

Aliquot (a)—The HSA ligand binding inhibitor, sodium octanoate, is added to the 1 ml of serum to a concentration of 100 mM as described in Example 4 above. The dye Diphenylhexatriene (DPH), which was dissolved in dimethyl formamide (DMF), was then slowly added under stirring to the sample to a final concentration of 0.4 mM. The sample was then excited at about 400 nm in order to cause the dye to fluoresce. The fluorescence was measured at an emission wavelength of about 440 nm, and from this value it was then possible to determine the concentration of total lipoprotein (HDL, LDL, and VLDL) in the sample.

Aliquot (b)—The HSA ligand binding inhibitor, sodium octanoate, is added to the 1 ml of serum to a concentration of 50 mM or 100 mM as described in Example 4 above. Furthermore benzoic acid may be added to the serum to a concentration of 5 mM. The probe Nile Red was then slowly added under stirring to the sample to a final concentration of 0.4 mM. The sample was then excited at 600 nm in order to cause the probe to fluoresce. The fluorescence was measured at an emission wavelength of 620 nm, and from this value it was then possible to determine the concentration of HDL in the sample as described in Example 5 above.

1. A method of determining the concentration of total lipoprotein in a sample, the method comprising the steps of:—(i) adding to an aliquot of the sample a lipophilic dye that binds to lipoproteins in the sample and which when so bound fluoresces under appropriate excitation; and (ii) determining the total lipoprotein concentration in the sample using fluorescence analysis.

2. The method of claim 1, wherein the lipophilic dye has two phenol groups that may be substituted or unsubstituted.

3. The method of claim 2, wherein the lipophilic dye is a chalcone dye.

4. The method of claim 3, wherein the chalcone dye is 4-methylaminomethylchalcone or K-37.

5. The method of claim 2, wherein the lipophilic dye is a dye comprising the fluorescent unit: Ph-[C—C═C]ₙ—C-Ph

6. The method of claim 5, wherein the dyes is diphenylhexatriene or diphenyloctatetrene.

7. The method of claim 1, wherein the dye is a Coumarin dye.

8. The method of claim 7, wherein the dye is Coumarin 30.

9. The method of claim 1, wherein the method further comprises adding to the aliquot a ligand binding inhibitor that substantially inhibits the binding of the dye to a hydrophobic binding domain on Human Serum Albumin before lipoprotein concentrations are determined.

10. The method of claim 9, wherein the ligand binding inhibitor comprises a fatty acid or a functional derivative thereof.

11. The method of claim 10, wherein the ligand binding inhibitor comprises octanoic acid (Cg) or a derivative thereof.

12. A method of analysing the lipoprotein content of a sample solution, the method comprising the steps of:—
(a) adding to a first aliquot of the sample a lipophilic dye that binds to lipoproteins in the sample and which when so bound fluoresces under appropriate excitation;
(b) determining the total lipoprotein concentration in the first aliquot using fluorescence analysis;
(c) adding to a second aliquot of the sample a discriminating dye that binds to a specific lipoprotein or lipoproteins in the sample and which when so bound fluoresces under appropriate excitation;
(d) determining the concentration of the lipoproteins in the second aliquot using fluorescence analysis; and
(e) calculating the lipoprotein content by comparing the concentrations determined in steps (b) and (d).

13. The method of claim 12, wherein the specific lipoprotein is HDL.

14. The method of claim 13, wherein the discriminating dye is Nile Red.

15. The method of claim 14, wherein the concentration of Nile Red added to the sample is between approximately 0.1-0.9 mM.

16. The method of claim 12, wherein the lipophilic dye is a dye selected from the group consisting of a calcone dye and a Coumarin dye.

17. The method of claim 12, wherein the method further comprises adding to the second aliquot a ligand binding inhibitor that substantially inhibits the binding of the dye to a hydrophobic binding domain on Human Serum Albumin before lipoprotein concentrations are determined.

18. The method of claim 12, wherein the sample is a biological fluid.

19. The method of claim 12, wherein the sample comprises blood plasma or serum, or lymph.

20. The method of claim 12, wherein the lipophilic dye is a dye selected from the group consisting of 4-methylaminomethylchalcone, K-37, diphenylhexatriene, diphenyloctatetrene, and Coumarin 30.

21. The method of claim 17, wherein the ligand binding inhibitor comprises a fatty acid or a functional derivative thereof.

22. The method of claim 21, wherein the ligand binding inhibitor comprises octanoic acid (Cg) or a derivative thereof.

23. The method of claim 1, wherein the sample is a biological fluid.

24. The method of claim 1, wherein the sample comprises blood plasma or serum, or lymph.
